(19) United States
(12) Patent Application Publication
(10) Pub. No.: US 2004/0146482 A1
(43) Pub. Date: Jul. 29, 2004

(54) Cosmetic and/or pharmaceutical preparations containing an extract of pterocarpus marsupium

(76) Inventors: Gilles Pauly, Nancy (FR); Christine Jeanmaire, Nancy (FR); Muriel Pauly-Florentiny, Nancy (FR)

Correspondence Address: COGNIS CORPORATION, PATENT DEPARTMENT, 300 BROOKSIDE AVENUE, AMBLER, PA, 19002, US

(21) Appl. No.: 10/479,134

(22) PCT Filed: May 23, 2002
(86) PCT No.: PCT/EP02/05659

(30) Foreign Application Priority Data
Jun. 1, 2001 (EP) 01401427.8

(51) Int. Cl. A61K 35/78; A61K 7/06

(52) U.S. Cl. 424/74; 424/725

(57) ABSTRACT

The invention relates to novel cosmetic and/or pharmaceutical preparations characterized in that they contain (a) an effective amount of an extract from Pterocarpus marsupium and (b) dicarboxylic acids and/or their salts and/or amino acids. The invention also relates to the use of said preparations as skin and/or hair care product, for instance against hair loss or skin aging.

### FIELD OF THE INVENTION

This invention relates generally to cosmetic and/or pharmaceutical products and, more particularly, to new preparations which, besides plant extracts of the plant Pterocarpus marsupium, contain dicarboxylic acids and/or salts thereof and/or amino acids and to the use of the preparations as skin- and/or hair-care preparations.

### PRIOR ART

Today, cosmetic preparations are available to the consumer in a variety of combinations. Consumers not only expect these preparations to have a certain care effect or to eliminate a certain deficiency, they are also increasingly demanding products which combine several properties and thus show an improved performance spectrum. Consumers are also entitled to expect the composition of the product to have optimal dermatological compatibility so that even consumers with sensitive skin do not react with irritation. In addition, the preparations are also expected to perform other functions which are related increasingly to care and particularly protection. There is a particular interest in substances which represent active principles that impart, for example, caring, anti-ageing and revitalizing properties to the skin and hair and, at the same time, positively influence, or at least do not adversely affect, the technical properties of the cosmetic product, such as storage stability, light stability and formulatability. In addition, consumers demand high dermatological compatibility and, above all, the use of natural products. Also, it is desirable to obtain distinctly better products by combining already known active principles or by discovering new applications for already known classes of substances. The effect of combining already known active components is often that positively synergistic effects are obtained and the concentration of active components to be used can be reduced. However, one disadvantage in this regard is often that a combination of active principles is only obtained when different plant extracts are simultaneously used in different quantity ratios.

Extracts of plants and their ingredients are being increasingly used in cosmetology and pharmacology. For many years, plant extracts have been used for medicinal purposes and also for cosmetic purposes in many different cultures. These plant extracts were often known only for very specific individual effects which limited their scope of application.

There is a growing interest in care components which combine pharmaceutical activity with minimal side effects, more especially in the border areas between cosmetology and pharmacology. If these care components are incorporated in cosmetic preparations, the consumer is able conveniently to eliminate or prevent deficiency symptoms without significant effort.

### DESCRIPTION OF THE INVENTION

The problem addressed by the present invention was to provide extracts of renewable raw materials for cosmetic and/or pharmaceutical application which, at the same time, would lend themselves to many uses as care components in various areas of cosmetology and/or pharmacology.

Another problem addressed by the invention was to provide cosmetic and/or pharmaceutical preparations which, besides care and protective properties, would above all have a preventive and curative effect, particularly on hair loss and on signs of ageing of the skin, and would be able to develop reactivating and revitalizing activity.

The present invention relates to cosmetic and/or pharmaceutical preparations containing

(a) an effective quantity of an extract of Pterocarpus marsupium and

(b) dicarboxylic acids and/or salts thereof and/or amino acids.

It has surprisingly been found that the use of extracts of Pterocarpus marsupium in combination with dicarboxylic acids and/or salts thereof and/or in combination with amino acids leads to products which combine favorable care and protective properties for the skin and particularly the scalp with high dermatological compatibility. The preparations thus obtained are distinguished by particularly good skin-care effects. The have a preventive and curative effect on hair loss and signs of skin ageing and a revitalizing and reactivating effect on the skin.

These multiple applications of the preparations according to the invention containing extracts of the renewable raw material of the plant Pterocarpus marsupium in combination with dicarboxylic acids and/or salts thereof and/or amino acids make it very attractive both to the market and to the consumer. Accordingly, the complex problem addressed by the invention has been solved by the use of these preparations.

In the context of the present invention, the term plant is understood to include both whole plants and plant parts (leaves, roots, flowers) and mixtures thereof.

Pterocarpus marsupium

The extracts according to the invention are obtained from plants of the Pterocarpus family. Actual examples are the extracts of Pterocarpus macrocarpus, P. santalinus (red sandalwood), P. angolensis, P. indicus, P. soyaauxii. From the performance perspective, however, the extract of Pterocarpus marsupium has proved to be particularly successful. The plant Pterocarpus marsupium is a plant which grows abundantly in leafy forests of Southern, Western and Eastern India and on Sri Lanka. It reaches a height of 15 to 25 meters. The dark-brown to gray bark shows large cracks and, after damage, secretes a reddish gum-like substance known as kino gum. The leaves are 15 to 25 cm in size and the flowers appear yellow. The fruits are flat and round and contain one or two small seeds. The heart of the wood is hard and golden to red-brown in color. In traditional Indian medicine, the wood and the bark are used inter alia as an anti-diabetic and anti-diarrheic. They also show astringent and anti-inflammatory activity. The leaves are used inter alia as an animal feed and also for healing wounds and particularly for curing skin diseases. The gum-like substance from the bark is used inter alia against blood diseases. J. Verghese reports on the general use of sandalwood extract in Cosm. Toil. 101(4), 69 (1986). The principal constituents of the Pterocarpus extracts are santalins, sterols and flavone derivatives. In this connection, reference is also made, for example, to French Patent FR-B1 2483228 (Pierre Fabre) which describes hair colorants based on Pterocarpus extracts. The use of Pterocarpus extracts as oxidative hair dyes is also described in Japanese patent application JP-A1 Hei 10/182372 (Lion). Finally, International patent application WO 98/44902 (L'Oréal) relates to self-tanning preparations containing sandalwood extract and a cosmetically acceptable oil component.

Extraction

The extracts to be used in accordance with the invention may be prepared by known methods of extracting plants or parts thereof. Particulars of suitable conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux in a Soxhlet extractor, which are familiar to the expert and which may all be used in principle, can be found, for example, in Hagers Handbuch der pharmazeutischen Praxis (5th Edition, Vol. 2, pp. 1026-1030, Springer Verlag, Berlin-Heidelberg-New York 1991). Fresh or dried plants or parts thereof are suitable as the starting material although plants and/or plant parts which may be mechanically size-reduced and optionally defatted before extraction are normally used. Any size reduction methods known to the expert, for example comminuting with a bladed tool, may be used.

Preferred solvents for the extraction process are water, organic solvents, water or mixtures of organic solvents and water, more particularly low molecular weight alcohols, esters, ethers, ketones or halogenated hydrocarbons with more or less large water contents (distilled or non-distilled), preferably aqueous alcoholic solutions with more or less large water contents. Extraction with distilled water, methanol, ethanol, propanol, butanol and isomers thereof, acetone, propylene glycols, polyethylene glycols, ethyl acetate, dichloromethane, trichloromethane and mixtures thereof is particularly preferred. The extraction process is generally carried out at 20 to 100° C., preferably at 80 to 100° C. and more particularly at 80 to 90° C. In one possible embodiment, the extraction process is carried out in an inert gas atmosphere to avoid oxidation of the ingredients of the extract. The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. If desired, the extracts thus prepared may be subjected, for example, to the selective removal of individual unwanted ingredients, for example by filtration. The extraction process may be carried out to any degree, but is usually continued to exhaustion. The present invention includes the observation that the extraction conditions and the yields of the final extracts may be selected according to the desired application. If desired, the extracts may then be subjected, for example, to spray drying or freeze drying. Typical yields (=extract dry matter, based on the quantity of raw material used) in the extraction of dried plants or dried plant parts (optionally defatted) are in the range from 1 to 20, preferably 2 to 15 and more particularly 3 to 10% by weight.

The quantity of plant extracts used in the preparations mentioned is governed by the concentration of the individual ingredients and by the way in which the extracts are used. In general, the total quantity of plant extract present in the preparations according to the invention is 0.001 to 25% by weight, preferably 0.005 to 10% by weight and more particularly 0.01 to 5% by weight, based on the final preparation, with the proviso that the quantities add up to 100% by weight with water and optionally other auxiliaries and additives.

The extracts according to the invention have an active substance content in the extracts of 5 to 100% by weight, preferably 10 to 95% by weight and more particularly 20 to 80% by weight. In the context of the invention, the active substance content is the sum total of all the active substances present in the extract, based on the dry weight of the extract.

Active substance in the context of the invention relates to the ingredients present in the extract even if their content and identity have yet to be established by conventional methods known to the expert. Active substances in the context of the invention are also any ingredients present in the extract of which the effect is either already known or has not yet been identified by conventional methods known to the expert.

Active substance in the context of the invention relates to the percentage content of substances and auxiliaries and additives present in the preparation except for the water additionally introduced.

The total content of auxiliaries and additives may be 1 to 50% by weight and is preferably 5 to 40% by weight, based on the final cosmetic and/or dermatological preparations. The preparations may be produced by standard cold or hot processes but are preferably produced by the phase inversion temperature method.

Extracts

In one preferred embodiment of the invention, the extracts of the plant Pterocarpus marsupium in the preparations according to the invention exclusively or predominantly contain flavone derivatives, more particularly kaempferol and kaempferol derivatives, 7-hydroxyflavanones, liquiritigenins, isoliquiritigenins, 7,44-dihydroxyflavones, marsupins, pterosurpins and (−)-epicatechol, as active substances. However, tannins and free phenolic acid, more particularly p-hydroxyphenyl lactic acid, may also be present as active substances in the extracts. The extracts differ in composition according to the starting material and extraction method selected.

Flavone derivatives in the context of the invention are understood to be those which can be isolated from the plant Pterocarpus marsupium.

More particularly, they are hydrogenation, oxidation or substitution products of 2-phenyl-4H-1-benzopyran; hydrogenation may already be present in the 2,3-position of the carbon chain, oxidation may already be present in the 4-position and substitution products are understood to be the replacement of one or more hydrogen atoms by hydroxy or methoxy groups. Accordingly, this definition also encompasses flavans, flavan-3-ols (catechols), flavan-3,4-diols (leucoanthocyanidines), flavones, flavonols and flavonones in the traditional sense. Particularly preferred flavone derivatives isolated from the plant Pterocarpus marsupium are kaempferol and kaempferol derivatives, 7-hydroxyflavanones, liquiritigenins, isoliquiritigenins, 7,44-dihydroxyflavones, marsupins, pterosurpins and (−)-epicatechol.

Besides the Pterocarpus marsupium extracts, the preparations according to the invention contain dicarboxylic acids and/or salts thereof and/or amino acids. A combination of Pterocarpus marsupium extract, salts of dicarboxylic acids and amino acids is particularly preferred.

Dicarboxylic Acids

In one preferred embodiment, the preparations according to the invention contain dicarboxylic acids selected from the group consisting of oxalic acid, malonic acid, succinic acid and glutaric acid. In another particular embodiment, the preparations according to the invention contain salts of these dicarboxylic acids selected from the group consisting of alkali metal and alkaline earth metal salts, more particularly sodium, potassium magnesium and calcium salts. Sodium succinate, the sodium salt of succinic acid, is particularly preferred.

Amino Acids

In one preferred embodiment, the preparations according to the invention contain amino acids selected from the group consisting of glycine, alanine, leucine, isoleucine, serine, threonine, cysteine, aspartic acid, glutamic acid, asparagine, glutamine, phenylalanine, tyrosine, methionine, valine, proline, lysine and histidine. Amino acids selected from the group consisting of histidine, aspartic acid and glutamic acid are particularly preferred, glutamic acid being most particularly preferred.

In one particular embodiment, the preparations according to the invention preferably contain

(a) 0.001 to 25% by weight extract and

(b) 0.001 to 15% by weight dicarboxylic acids and/or salts thereof, with the proviso that the quantities shown optionally add up to 100% by weight with water and/or other auxiliaries and additives.

In another particular embodiment, the preparations preferably contain

(a) 0.001 to 25% by weight extract and

(b) 0.0005 to 10% by weight amino acids,

with the proviso that the quantities shown optionally add up to 100% by weight with water and/or other auxiliaries and additives.

In another particular embodiment, the preparations according to the invention preferably contain

(a) 0.001 to 25% by weight extract and

(b) 0.001 to 15% by weight dicarboxylic acids or salts thereof and 0.0005 to 10% by weight amino acids,

with the proviso that the quantities shown optionally add up to 100% by weight with water and/or other auxiliaries and additives.

The combination of Pterocarpus marsupium extracts with dicarboxylic acids and/or salts thereof and/or amino acids and in particular the active substances from the extracts, the flavone derivatives, show a large number of cosmetic and pharmaceutical effects.

Accordingly, the present invention also relates to the use of preparations containing

(a) an effective quantity of an extract of Pterocarpus marsupium and

(b) dicarboxylic acids and/or salts thereof and/or amino acids

[symbol] as hair and skin care preparations,

[symbol] against hair loss, more particularly against androgenic alopecia;

[symbol] as preparations with anti-glycosylation activity, more particularly against the glycosylation of cutaneous proteins and preferably against the glycosylation of collagen; elastin; GAGs and small proteoglucans, such as decorin and biglucan;

[symbol] as active components for stimulating the synthesis of glutathiones;

[symbol] as active anti-inflammatory components;

[symbol] as active components for reducing the formation of interleucine-1-alpha (1′IL-α);

[symbol] as active components against the ageing of skin;

[symbol] as UV/IR protection factors;

[symbol] as active components against the damaging of fibroblasts and/or keratinocytes by UV radiation, more particularly UV-B radiation;

[symbol] as active components against oxidative skin and/or hair stress;

[symbol] in protective and restorative care preparations with revitalizing and reactivating activity for the skin and especially the scalp.

Although the synergistic combination of the Pterocarpus marsupium extracts with dicarboxylic acids and/or salts thereof and/or amino acids is preferred and shows particularly good effects in the applications mentioned, the Pterocarpus marsupium extracts show a large number of cosmetic and pharmaceutical effects even without the synergistic combination. Accordingly, the present invention also relates to the use of Pterocarpus marsupium extracts

[symbol] against hair loss, more particularly against androgenic alopecia;

[symbol] as preparations with anti-glycosylation activity, more particularly against the glycosylation of cutaneous proteins and preferably against the glycosylation of collagen; elastin; GAGs and small proteoglucans;

[symbol] as active components for stimulating the synthesis of glutathiones;

[symbol] as active components for reducing the formation of interleucine-1-alpha (1′IL-α);

[symbol] as active components against the ageing of skin;

[symbol] as UV/IR protection factors;

[symbol] as active components against the damaging of fibroblasts and/or keratinocytes by UV radiation, more particularly UV-B radiation;

[symbol] as active components against oxidative skin and/or hair stress;

[symbol] in protective and restorative care preparations with revitalizing and reactivating activity for the skin and especially the scalp.

Care Preparations

Care preparations in the context of the invention are understood to be care preparations for the hair and the skin, especially the scalp. These care preparations include inter alia stimulating, healing and restorative activity. Preferred care preparations in the context of the invention are care preparations active against hair loss which have a stimulating effect on the skin cells and their functions and which also have a restorative effect on skin and hair. In addition, they have a preventive effect against environmental influences on the skin. Other preferred care preparations in the context of the invention are those which can either improve or cure various skin diseases through their various effects on the appearance and function of the skin. In principle, the extracts according to the invention may be used in any cosmetic products for topical application. Examples of cosmetic products and their formulations can be found in Tables 5 to 8.

The present invention includes the observation that particularly effective cosmetic preparations are obtained through the co-operation of the ingredients of the plant extracts, particularly those mentioned above.

The preparations according to the invention combine excellent skin care activity with high dermatological compatibility. In addition, they show high stability, particularly to oxidative decomposition of the products.

In the context of the invention, the terms “preparations” and “final preparations” are synonymous with the term “care preparations”.

Active substance in the context of the invention relates to the percentage content of substances and auxiliaries and additives present in the preparation except for the water additionally introduced.

Hair Loss

The preparations according to the invention and Pterocarpus marsupium extracts are active against hair loss and particularly against androgenetic hair loss which is also known as androgenic alopecia.

A hair loss of 30-100 hairs/day is regarded as the physiological norm.

The hair loss which leads to baldness in men and women is predominantly hormonal in origin (so-called androgenic alopecia) although genes do play a part. In men, baldness begins with the appearance of a receding hairline. Androgenic alopecia is characterized by certain changes in the hair follicles. These changes include a progressive reduction in the size of the hair follicles, a shortening of the growth phase of the hair (anagen phase) and a lengthening of the quiescent phase (telogen phase). This suggests that androgenic alopecia is a complex phenomenon. Various studies have already been conducted with a view to reducing or preventing this phenomenon.

For example Reygagne et al. investigated the influence of 5-alpha-reductase inhibitors (Annales Dermatol. Venerol. 3, 1998) on alopecia while Lachgar et al. attempted to increase the expression of VEGF (vascular endothelial growth factor) with Minoxidil and thus to achieve an increase in the formation of blood vessels in the hair follicles (British Journal of Dermatology, 138, 1998, 407-411). According to FR 2724561, alopecia can be controlled by the use of lysyl hydroxylase inhibitors (lysyl hydroxlyase is an enzyme of collagen development).

Androgenic alopecia is different from the morbid forms of hair loss (effluvium) which occur inter alia in Alopecia areata, a disease where bald patches of skin are caused presumably by autoimmunity, or with various infectious diseases (syphilis, fungal diseases).

Telogenic hair loss can be caused by infections or can occur after pregnancy. It can also be caused by disease-modified hair growth. Hair loss is also possible in cases of malnutrition, after application of medicaments (for example cytostatic agents, anticoagulants) and in the event of poisoning (for example with thallium salts).

It was possible to demonstrate that the enzymatic glycosylation of proteins in the skin and the scalp can be detected although the level of AGEs (advanced glycating end products), as measured in one and the same person, is higher in the tissues of hairless areas than in hair-covered areas.

The preparations and Pterocarpus marsupium extracts according to the invention show antiglycosylation activity and, in particular, are active against the glycosylation of cutaneous proteins and preferably against the glycosylation of collagen. In 1981, A. Cerami (Science, 1981, 211, 491-493) described the glycosylation of proteins or non-enzymatic glysolyation as opposed to enzymatic glycosylation by glucosyl transferase and mentioned the possible role of this glycosylation in the ageing of tissue. The biochemical mechanism of this reaction is well known (Borel J. P. et al., CR biologie prospective, 145-149, 1993) and comprises two phases:

In an early phase, reducing sugars (glucose, fructose) react with the terminal or lateral amino functions of the proteins present in tissue to form so-called Schiffs bases. These compounds are then stabilized by Amadori rearrangement to the ketoamine.

In a late phase, the ketoamine functions are then oxidized in the presence of oxygen to form deoxyonose and react with other basic amino acids, such as arginine or lysine, belonging to other proteins (albumin, lipoproteins, immunoglobulin). This results in the formation of complexes which are ultimately bridged through pentosidine or 2-furoyl-1,4-imidazole cycles. The so-called AGEs (advanced glycosylation end products) are formed as complex and highly stable end products of this bridging. This late phase is very slow and irreversible.

The glycosylation of the proteins leads to the formation of inter- and intramolecular bridges in slowly renewed proteins and ultimately to the brown coloration and insolubility of these proteins. Glycosylation particularly affects the proteins in the extracellular matrices of which the renewal is slow.

In the case of the skin, the proteins damaged by glycosylation are in particular fibronectin, laminin, elastin, various collagen types, elastin, GAGs and small proteoglucans.

The AGEs lead to various complaints:

15. because they are bulky, the molecules which carry them have difficulty in remaining in their normal place,

16. the glycosylated molecules lose their flexibility (tissue stiffening),

17. the glycosylated molecules can become more resistant to enzymes which guarantee their renewal and thus form areas of amorphous substances.

In normal skin, the glycosylated proteins are eliminated via the metabolism and the cells, more particularly through degradation by macrophages which induces re-formation of the dermis.

However, this elimination diminishes with increasing age, resulting in the accumulation of these glycosylated proteins and in accelerated and increased ageing of the dermis for which several phenomena together are responsible.

18. resistance to renewal proteases and a decrease in fibrillogenesis and hence in the renewal of collagen, a reduction in its filter effect in the extracellular matrix and more particularly at the dermis/epidermis boundary after fixing of foreign proteins (LDL, cholesterol, albumin) which leads to thickening,

19. the glycosylated proteins represent a potential source of free oxygen radicals. This phenomenon—intensified by UV-A—leads to collagen degradation,

20. activation of non-specific harmful proteases,

21. activation of macrophages and dumping of cytokinins (TNF-alpha),

22. finally inflammation with subsequent fibrosis and deposition of lipofuszin.

The foregoing observations are of interest for the use of substances with anti-glycosylation activity, more particularly in the control and prevention of ageing of the skin and especially the hair in the cosmetics field. The suppression of non-enzymatic glycosylation is inter alia an important objective in the prevention of hair loss.

Studies have shown that high exposure of the skin to UV radiation leads to an increase in the glycosylation of certain proteins. Since the scalp is particularly exposed to UV radiation and since glycosylation correlates directly with the advance of hair loss, preparations which counteract glycosylation may be directly used against hair loss. If such preparations additionally act as UV/IR protection factors, as the preparations according to the invention have been shown to do, they can be said to have the desired manifold effect.

Another effect which can lead to an increase in glycosylation is the reduction of glutathione. The preparations according to the invention and Pterocarpus marsupium extracts have a stimulating effect on the synthesis of glutathione. Glutathione is an important tripeptide for the metabolism, the transport and the protection of the cell. It is known that the content of glutathione in the hair follicles decreases with age. In addition, studies have already shown that the content of glutathione in the scalp cells affected by hair loss was lower than in the regions of the scalp still covered with hair (Giralt et al. J. Invest. Dermatol. 107, 1996, 154-158). Accordingly, the stimulating effect of the preparations according to the invention on the synthesis of glutathione can lead indirectly to a reduction of hair loss.

The preparations according to the invention also show amti-inflammatory activity. The preparations according to the invention may be used in principle as anti-inflammatory additives in any cosmetic and/or pharmaceutical care products used against inflammation of the skin and hence in skin care. Anti-inflammatory care preparations in the context of the invention are care preparations of the kind which are capable of curing or preventing inflammation of the skin. The inflammation can be caused by various factors.

The preparations according to the invention and Pterocarpus marsupium extracts also reduce the formation of interleucine-1-alpha (1′IL-α).

1′IL-α is a mediator of the immune system belonging to the cytokinins which is capable of influencing the growth (as growth factors), differentiation and activity of cells (immunomodulation).

Specifically, 1′IL-α is involved in the anagen phase of the growth of hair follicles and presumably initiates the transition to the catagen phase. Studies have shown that 1′IL-α can inhibit hair growth. A high concentration of 1′IL-α leads to inflammatory reactions. Accordingly, there is a connection between the concentration of 1′IL-α, hair loss and inflammatory reactions. The formation of 1′IL-α is reduced and hence inflammatory reactions are indirectly avoided and inter alia hair loss prevented by the preparations according to the invention and by Pterocarpus marsupium extracts.

The preparations according to the invention and Pterocarpus marsupium extracts are also active against signs of skin ageing and may be used for the preventive or curative treatment of ageing of the skin. Care preparations of this kind are also known as anti-ageing preparations. These signs of ageing include, for example, lining and wrinkling of any kind. The treatments include the slowing down of skin ageing processes. The signs of ageing can be caused by various factors. The ageing process can be induced by UV radiation and, in that case, is known as photoageing. In one particular embodiment of the invention, these care preparations are used for the treatment of UV-induced signs of skin ageing.

The preparations according to the invention and Pterocarpus marsupium extracts act as UV protection factors.

Sun (UV) Protection Factors

Sun protection factors or UV protection factors in the context of the invention are light protection factors which are useful in protecting human skin against harmful effects of direct and indirect solar radiation. The ultraviolet radiation of the sun responsible for tanning of the skin is divided into the sections UV-C (wavelengths 200-280 nm), UV-B (280-315 nm) and UV-A (315-400 nm).

The pigmenting of normal skin under the influence of solar radiation, i.e. the formation of melanins, is differently effected by UV-B and UV-A. Exposure to UV-A (long-wave UV) results in darkening of the melanins already present in the epidermis without any sign of harmful effects. It is different with so-called short-wave UV (UV-B). This promotes the formation of so-called late pigment through the reformation of melanins. However, before the (protective) pigment is formed, the skin is exposed to the unfiltered radiation which, depending on the exposure time, can lead to reddening of the skin (erythema), inflammation of the skin (sunburn) or even blisters.

These other UV protection factors are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:

[symbol] 3-benzylidene camphor or 3-benzylidene norcamphor and derivatives thereof, for example 3-(4-methylbenzylidene)-camphor as described in EP-BI 10693471;

[symbol] 4-aminobenzoic acid derivatives, preferably 4-(dimethylamino)benzoic acid-2-ethylhexyl ester, 4-(dimethylamino)-benzoic acid-2-octyl ester and 4-(dimethylamino)-benzoic acid amyl ester;

[symbol] esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);

[symbol] esters of salicylic acid, preferably salicylic acid-2-ethylhexyl ester, salicylic acid-4-isopropylbenzyl ester, salicylic acid homomenthyl ester;

[symbol] derivatives of benzophenone, preferably 2-hydroxy-4-methoxybenzo-phenone, 2-hydroxy4-methoxy4′-methylbenzophenone, 2,2′-dihydroxy-4-methoxybenzophenone;

[symbol] esters of benzalmalonic acid, preferably 4-methoxybenzmalonic acid di-2-ethylhexyl ester;

[symbol] triazine derivatives such as, for example, 2,4,6-trianilino-(p-carbo-2′-ethyl-1′-hexyloxy)-1,3,5-triazine and Octyl Triazone as described in EP 0818450 A1 or Dioctyl Butamido Triazone (UVAsorb® HEB);

[symbol] propane-1,3-diones such as, for example, 1-(4-tert.butylphenyl)-3-(4′-methoxyphenyl)-propane-1,3-dione;

[symbol] ketotricyclo(5.2.1.0)decane derivatives as described in EP 0694521 B1.

Suitable water-soluble substances are

[symbol] 2-phenylbenzimidazole-5-sulfonic acid and alkali metal, alkaline earth metal, ammonium, alkylammonium, alkanolammonium and glucammonium salts thereof;

[symbol] sulfonic acid derivatives of benzophenones, preferably 2-hydroxy-4-methoxybenzophenone-5-sulfonic acid and salts thereof;

[symbol] sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UVA filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and the enamine compounds described in DE 19712033 A1 (BASF). The UV-A and UV-B filters may of course also be used in the form of mixtures. Particularly favorable combinations consist of the derivatives of benzoyl methane, for example 4-tert-butyl-4′-methoxydibenzoylmethane (Parsol® 1789) and 2-cyano-3,3-phenylcinnamic acid-2-ethyl hexyl ester (Octocrylene) in combination with esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof. Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or dimethicones. So-called micro- or nanopigments are preferably used in sun protection products. Micronized zinc oxide is preferably used. Other suitable UV filters can be found in P. Finkel's review in SÖFW-Journal 122, 543 (1996) and in Parfümerie und Kosmetik 3 (1999), pages 11 et seq.

The preparations according to the invention and Pterocarpus marsupium extracts are also used against the damage to fibroblasts and/or keratinocytes by UV radiation.

UVB rays initiate inflammation by activating an enzyme, namely phospholipase A2 or PLA2. This inflammation (erythema, edema) is induced by the removal of arachidonic acid from the phospholipids of the plasma membrane by the phospholipase. Arachidonic acid is the precursor of the prostaglandins which cause inflammation and cell membrane damage. The prostaglandins E2 (=PGE2) are formed by cyclooxygenase. The degree of release of the cytoplasm enzyme LDH (lactate dehydrogenase) in human keratinocytes serves as a marker for cell damage.

The preparations according to the invention reduce the effect of UV-B radiation on the number of keratinocytes and on the content of released LDH. Accordingly, the preparations and extracts have the ability to reduce cell membrane damage caused by UV-B radiation.

The preparations according to the invention and Pterocarpus marsupium extracts may be used against oxidative stress for the skin and/or hair. Besides the preparations according to the invention and Pterocarpus marsupium extracts, antioxidants and radical traps may also be used against oxidative stress.

Antioxidants are capable of inhibiting or preventing unwanted changes caused by the effects of oxygen and other oxidative processes in the substances to be protected. The effect of antioxidants consists mainly in their acting as radical traps for the free radicals occurring during autoxidation.

One possible use of the antioxidants, for example in cosmetic and/or dermatological preparations, is their use as secondary sun protection factors because antioxidants are capable of interrupting the photochemical reaction chain which is initiated when UV rays penetrate into the skin. Besides the plant extract according to the invention, other typical examples are amino acids (for example glycine, alanine, arginine, serine, threonine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene, lutein) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, boldin, boldo extract, EDTA, EGTA and derivatives thereof, unsaturated fatty acids and derivatives thereof (for example y-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

The UV protection factors or antioxidants may be added in quantities of 0.01 to 25, preferably 0.03 to 10 and more particularly 0.1 to 5% by weight, based on the total quantity in the preparations.

The preparations according to the invention and Pterocarpus marsupium extracts may also be used in protective and restorative care preparations with skin revitalizing and reactivating activity. This way of using the care preparations has a positive effect, for example, on the adverse effects of environmental contamination of the skin by reactivating the natural functions of the skin and by making the skin more resistant. In principle, the extracts according to the invention may be used as protective and restorative care preparations for any preparations which are used to prevent damage or to treat damage to the skin and hence in skin care. Another use in this field is application to sensitive skin damaged by allergies or other factors. The skin damage can have various causes.

The preparations according to the invention may be used for the production of cosmetic and/or dermatological preparations such as, for example, shampoos, hair rinses, hair treatments, shower baths, creams, gels, lotions, alcohol and water/alcohol solutions, emulsions, wax/fat compounds, stick preparations, powders or ointments. These preparations may additionally contain mild surfactants, oil components, emulsifiers, pearlizing waxes, consistency factors, thickeners, superfatting agents, stabilizers, polymers, silicone compounds, fats, waxes, lecithins, phospholipids, biogenic agents, anti-dandruff agents, film formers, swelling agents, insect repellents, self-tanning agents, tyrosinase inhibitors (depigmenting agents), hydrotropes, solubilizers, preservatives, perfume oils, dyes and the like as further auxiliaries and additives.

Surfactants

Suitable surfactants are anionic, nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöadditive (Catalysts, Surfactants and Mineral Oil Additives)”, Thieme Verlag, Stuttgart, 1978, pages 123-217. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Oil Components

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂ fatty alcohols, esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₈₋₃₈ alkylhydroxycarboxylic acids with linear or branched C₆₋₂₂ fatty alcohols (cf. DE 19756377 A1), more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di-and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates such as, for example, Dicaprylyl Carbonate (Cetiol® CC), Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group such as, for example, Dicaprylyl Ether (Cetiol® OE), ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

Emulsifiers

Suitable emulsifiers are, for example, nonionic surfactants from at least one of the following groups:

[symbol] products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and alkylamines containing 8 to 22 carbon atoms in the alkyl group;

[symbol] alkyl and/or alkenyl oligoglycosides containing 8 to 22 carbon atoms in the alkyl group and ethoxylated analogs thereof;

[symbol] addition products of 1 to 15 mol ethylene oxide onto castor oil and/or hydrogenated castor oil;

[symbol] addition products of 15 to 60 mol ethylene oxide onto castor oil and/or hydrogenated castor oil;

[symbol] partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 mol ethylene oxide;

[symbol] partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 mol ethylene oxide;

[symbol] mixed esters of pentaerythritol, fatty acids, citric acid and fatty alcohol according to DE 1165574 PS and/or mixed esters of fatty acids containing 6 to 22 carbon atoms, methyl glucose and polyols, preferably glycerol or polyglycerol,

[symbol] mono-, di- and trialkyl phosphates and mono-, di- and/or tri-PEG-alkyl phosphates and salts thereof,

[symbol] wool wax alcohols,

[symbol] polysiloxane/polyalkyl/polyether copolymers and corresponding derivatives,

[symbol] block copolymers, for example Polyethyleneglycol-30 Dipolyhydroxystearate;

[symbol] polymer emulsifiers, for example Pemulen types (TR-1, TR-2) of Goodrich;

[symbol] polyalkylene glycols and

[symbol] glycerol carbonate.

The addition products of ethylene oxide and/or propylene oxide with fatty alcohols, fatty acids, alkylphenols or with castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol are known as lipid layer enhancers for cosmetic formulations from DE 2024051 PS.

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide with the partial glycerides mentioned are also suitable.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxy-stearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide with the sorbitan esters mentioned are also suitable.

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Poly-glyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 mol ethylene oxide.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Fats and Waxes

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquato (BASF), condensation products of polyglycols and amines, quaternized collagen polypeptides such as, for example, Lauryldimonium Hydroxypropyl Hydrolyzed Collagen (Lamequat® L, Grünau), quaternized wheat poly-peptides, polyethyleneimine, cationic silicone polymers such as, for example, Amodimethicone, copolymers of adipic acid and dimethylamino-hydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2252840 A and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamido-propyl trimethylammonium chloride/acrylate copolymers, octylacryl-amide/methyl methacrylate/tert.-butylaminoethyl methacrylate/2-hydroxy-propyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosm. Toil. 108, 95 (1993).

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Biogenic Agents

Biogenic agents in the context of the invention are additionally those which do not come from the plant Pterocarpus marsupium such as, for example, tocopherol, tocopherol acetate, tocopherol palmitate, ascorbic acid, deoxyribonucleic acid and fragmentation products thereof, retinol, bisabolol, allantoin, phytantriol, panthenol, AHA acids, ceramides, pseudoceramides, essential oils, other plant extracts and additional vitamin complexes.

Antidandruff Agents

Suitable antidandruff agents are Pirocton Olamin (1-hydroxy4-methyl-6-(2,4,4-trimethylpentyl)-2-(1H)-pyridinone monoethanolamine salt), Baypival® (Climbazole), Ketoconazol® (4-acetyl-1 -{4-[2-(2,4-dichlorophenyl) r-2-(1H-imidazol-1-ylmethyl)-1,3-dioxylan-c-4-ylmethoxy-phenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione and magnesium pyrithione/dipyrithione magnesium sulfate.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Swelling Agents

Suitable swelling agents for aqueous phases are montmorillonites, clay minerals, Pemulen and alkyl-modified Carbopol types (Goodrich). Other suitable polymers and swelling agents can be found in R. Lochhead's review in Cosm. Toil. 108, 95 (1993).

Insect Repellents

Suitable insect repellents are N,N-diethyl-m-toluamide, pentane-1,2-diol or Ethyl Butylacetylaminopropionate.

Self-Tanning Agents and Depigmenting Agents

A suitable self-tanning agent is dihydroxyacetone. Suitable tyrosinase inhibitors which prevent the formation of melanin and are used in depigmenting agents are, for example, arbutin, ferulic acid, koji acid, coumaric acid and ascorbic acid (vitamin C).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are

[symbol] glycerol;

[symbol] alkylene glycols such as, for example, ethylene glycol, diethylene glycol, propylene glycol, butylene glycol, hexylene glycol and polyethylene glycols with an average molecular weight of 100 to 1000 dalton;

[symbol] technical oligoglycerol mixtures with a degree of self-condensation of 1.5 to 10 such as, for example, technical diglycerol mixtures with a diglycerol content of 40 to 50% by weight;

[symbol] methylol compounds such as, in particular, trimethylol ethane, trimethylol propane, trimethylol butane, pentaerythritol and dipenta-erythritol;

[symbol] lower alkyl glucosides, particularly those containing 1 to 8 carbon atoms in the alkyl group, for example methyl and butyl glucoside;

[symbol] sugar alcohols containing 5 to 12 carbon atoms, for example sorbitol or mannitol,

[symbol] sugars containing 5 to 12 carbon atoms, for example glucose or sucrose;

[symbol] amino sugars, for example glucamine;

[symbol] dialcoholamines, such as diethanolamine or 2-aminopropane-1,3-diol.

Preservatives

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”).

Perfume Oils

Suitable perfume oils are mixtures of natural and synthetic fragrances. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Dyes

Suitable dyes are any of the substances suitable and approved for cosmetic purposes as listed, for example, in the publication “Kosmetische Fäirbemittel” of the Farbstoffkommission der Deutschen Forschungs-gemeinschaft, Verlag Chemie, Weinheim, 1984, pages 81 to 106. These dyes are normally used in concentrations of 0.001 to 0.1% by weight, based on the mixture as a whole.

### EXAMPLES

### 1. Example. Extraction of the Plants with Distilled Water

20 liters distilled water were heated to 80° C. and 4.5 kg crushed Pterocarpus marsupium plants were added. The mixture was intensively stirred for 2 h, cooled to room temperature and then centrifuged for 10 mins. at a speed of 3500 G. The supernatant liquid (extract) was red in color. To remove insoluble impurities, the extract was filtered, frozen over a period of 72 h and then dried either by freeze-drying or by spray-drying. For spray-drying, the extract was spray-dried at a starting temperature of 185° C. and a final temperature of 80° C. The yield of extract was 3 to 10% by weight, based on the weight of plants used.

Another method of working up comprises fractionating the extract, for example by conventional methods of chromatography. The yield of the fraction containing flavone derivatives was 0.005 to 0.5% by weight, based on the dry weight of plants used.

### 2. Example. Extraction of the Plants with Aqueous Methanol

Example 1 was repeated except that extraction was carried out with 20 liters 96% by weight ethanol. Extraction was carried out and the extract was further processed in the same way as described in Example 1. Filtration was also carried out as described in Example 1. Thereafter, the alcohol was removed under reduced pressure at 40° C. and replaced by distilled water. To remove insoluble impurities, the extract was filtered and frozen over a period of 72 h and then either freeze-dried or spray-dried. The yield of extract was 2 to 8% by weight, based on the dry weight of plants used. The yield of fraction containing flavone derivatives was 0.005 to 0.5% by weight, based on the dry weight of plants used.

3. Toxicity Test

The object of this test is to determine the toxic concentration of the preparation to be studied for fibroblasts in order better to determine the most effective concentration. The effective concentration range can thus be narrowed.

The preparation to be studied has the following composition (% by weight):

| | |
|---|---|
| Pterocarpus marsupium extract | 5.00% |
| sodium succinate | 2.50% |
| glutamic acid | 0.50% |
| mannitol | 92.00% |

It is obtainable, for example, under the name of Trichodyn®. In the interests of simplicity, the following Examples refer to Trichodyn®, but what is meant are preparations according to the invention having the above-mentioned composition.

Method: effects on cell growth. Human fibroblasts were inoculated with 10% by weight of fetal calf serum in a defined nutrient medium (DMEM=Dulbecco Minimum Essential Medium, a product of Life Technologie S.a.r.l.) and incubated for 24 h at 37° C. in a 5% CO₂ atmosphere. The nutrient medium containing fetal calf serum was then replaced by a nutrient medium of DMEM without fetal calf serum. Active substance in the form of the above-mentioned composition Trichodyn® was then added to this nutrient medium in various concentrations. After the fibroblasts had been incubated for three days in the nutrient medium, growth and metabolic activity were evaluated by determining the intracellular content of ATP by Vasseur's enzymatic luminescence method (Journal Français Hydrologie, 1981, 9, 149-156) and the cell protein content by Bradford's method (Anal. Biochem., 1976, 72, 28-254).

Determining the cell protein content provides an indication of the number of macromolecules, such as enzymes, collagen, elastin or other dermal macromolecules, which is required for forming connective tissue. The ATP content of a cell is important for many enzymes whose activity is dependent upon this energy carrier.

The lethal Trichodyn® dose at which 50% of the fibroblasts studied were no longer viable was determined (LD 50). Up to a concentration of 0.1% by weight, Trichodyn® does not have a toxic effect on the human fibroblasts. In the determination of the protein contents, the LD 50 was at 0.53%. In the determination of ATP, this value was determined for a Trichodyn® concentration of 0.67% by weight.

TABLE 1
Determination of the toxic Trichodyn ® concentration by determining
the protein content and the ATP content in human fibroblasts

| | Proteins | ATP |
|---|---|---|
| LD 50 (% by weight) | 0.53 | 0.67 |

4. Viability

Method 2: improvement of viability. The test was carried out on human fibroblasts. It enables a certain number of parameters to be quantitatively determined on the resting cells. The cultivation of the cells corresponds to the cultivation of in the method described above except for the incubation time. The incubation time for this test was 72 h. Viability was evaluated by determination of the sulfur-rich protein metallothionein (MTT); by calorimetric determination of the percentage protein content by Bradford's method (Anal. Biochem. 1976, 72, 248-254); and by determination of the percentage glutathione content (GSH) with a fluorescent probe, orthophthaldehyde, by Hissin and Hilf's method (Anal. Biochem. 1976, 74, 214-216). The glutathione (GSH) is produced by cells in order to be able to react directly against oxidative stress and environmental influences, such as high heavy metal levels. The three amino acids that are present in bound form in the glutathione are synthesized by specific enzymes which need ATP. Accordingly, an increased percentage content of GSH after treatment of the cells with Trichodyn® is a measure of the increased viability of the cells under the effect of external stress and other challenges.

The tests were carried out three times and then repeated twice. The results were expressed in percent by comparison with the control.

TABLE 2
Determination of the protein content
and GSH content in human fibroblasts

| Concentration in | | Protein | GSH |
|---|---|---|---|
| % by weight | MTT | content in % | content in % |
| 0 | 100 | 100 | 100 |
| 0.6 | 90 | 92 | 134 |

The results show that Trichodyn® in a concentration of 0.6% by weight increases the percentage content of cellular glutathione and may therefore be used inter alia against oxidative stress for skin or hair.

5. Cell Protecting Effect Against UV-B in Human Keratinocytes Cultivated in vitro

Background: UV-B rays cause inflammation (erythema, edema) by activating an enzyme, namely phospholipase A2 or PLA2, which removes arachidonic acid from the phospholipids of the cell membrane. Arachidonic acid is the precursor of the prostaglandins which cause inflammation and cell membrane damage; the prostaglandins E2 (=PGE2) are formed by cyclooxygenase. A UV-challenged cell produces another inflammation mediator, namely interleucine-1-alpha (1′ILα), which is capable of inducing the secretion of prostaglandins.

Method: The effect of UV-B radiation was investigated in vitro in keratinocytes by determining the release of the cytoplasm enzyme LDH (lactate dehydrogenase) and the(1′ILα) content.

To carry out the tests, a defined medium containing fetal calf serum was inoculated with the keratinocytes and Trichodyn® was added 72 hours after the inoculation.

The keratinocytes were then exposed to a dose of UV-B (50 mJ/cm²-tubes: DUKE GL40E).

After incubation for another day at 37° C./5% CO₂, the LDH content in the supernatant was determined. The LDH (lactate dehydrogenase) content was determined by an enzyme reaction (kit used to determine LDH levels from Roche). In addition , the (1′ILα) content was determined by the ELISA test. The number of adhering keratinocytes was determined (after trypsin treatment) with a particle counter.

TABLE 3
Cell protecting effect against UV-B rays; results in % based
on the control, mean value of 2 tests each repeated twice

| | | Content | |
|---|---|---|---|
| | No. of | of released | (1′ILα) |
| | keratinocytes | LDH | content |
| Control without UV-B | 100 | 0 | 0 |
| UV-B | 17 | 100 | 100 |
| UV-B + Trichodyn ® 0.1% | 26 | 58 | 70 |
| UV-A + Trichodyn ® 0.2% | 26 | 43 | 72 |
| UV-B + aspirin | 15 | 66 | 69 |

The results of these tests show that a preparation according to the invention reduces the effect of UVB radiation on the number of keratinocytes, on the (1′ILα) content and on the content of LDH released. Accordingly, the described preparations have the ability to reduce the damage to cell membranes caused by UV-B radiation.

6. Example. Anti-Glycosylation Test on Type I Collagen

Background: The non-enzymatic glycosylation of the proteins is a critical process in the ageing of human tissue and explains the reticulation of the extracellular matrix and the basal membrane. The glycosylation at dermal proteins with reducing sugars, such as glucose, fructose or sucrose, results in the formation of long-lived Schiff's bases. This mechanism destroys the extracellular matrix structure and the metabolism of the fibroblasts. In addition, the Schiff s bases catalyze the production of reactive forms of oxygen which can further intensify the effects of the non-enzymatic glycosylation, particularly by UV-A radiation. In addition, oxidative stress promotes stabilization of the Schiff's bases up to the so-called AGE's (advanced glycated end products) which accumulate in the skin.

Method: The in-tubo test was carried out on type I collagen which was incubated for 21 days at 45° C. in the presence of 1% glycose. The content of Schiffs bases was determined by fluorescence at 430 nm (excitation at 350 nm) on the first and last day of the 21-day incubation period.

TABLE 4
Fluorescence determination of collagen
after 21 days incubation with glucose

| | Fluorescence/ |
|---|---|
| | control on day 21 |
| Control without glucose | 47 |
| Control with glucose | 100 |
| Glucose + Trichodyn ® (0.3% by wt.) | 81 |
| Glucose + Trichodyn ® (1% by wt.) | 36 |
| Glucose + Trichodyn ® (3% by wt.) | 24 |

The results impressively demonstrate that Trichodyn® clearly reduces the glycosylation of type I collagen. Even in a concentration of 0.3% by weight, glycosylation is reduced by comparison with the control with glucose. The IC 50 value (Trichodyn® concentration at which 50% glycosylation was inhibited) was determined as 0.4% by weight.

7. Clinical Study

The clinical study was conducted with 30 male volunteers with diffuse androgenic alopecia (types I to III). 15 volunteers were treated with placebo lotion, 15 volunteers received lotion containing 10% active ingredient (Trichodyn®). The selection test consisted of a trichogram in which the A/T ratio (hair in the anagenic phase/hair in the telogenic phase) had to be under 3 in at least one region of the test scalp.

Method:

10 ml of the lotion was rubbed into the scalp and hair three times a week for 6 months.

The anti-hair loss activity was quantitatively determined by a thrichogram according to Bouhann, P., Reygagne, P.: Pathologie du cheveu. Masson, Paris, 1999. The trichogram was recorded over two regions of the scalp (frontal and occipital) before the treatment and then after 2, 4 and 6 months, 30 to 50 hair fibers being subjected to standardized sampling to determine the percentage of telogenic hairs (T). The development of telogenic hairs was determined in % in relation to the basic value derived from the mean value of the determination in the particular volunteer group and on the particular scalp area (frontal, occipital) on day 0.

It is important in this connection to bear in mind that hair loss is also season-dependent. In February and March, hair loss is lower and then increases from March to the end of July (Randall, V. A., Ebling, F. J. G.: Seasonal changes in human hair growth. British Journal of Dermatology, 124: 146-151, 1991). The clinical test began in the phase of low hair loss and ended in the highly active telogenic phase.

The reduction in the percentage content of telogenic hairs corresponds with the reduction in hair loss activity. Product tested (quantities in % by weight, based on the formulation):

| | |
|---|---|
| Water | 78.78 |
| Active ingredient (Trichodyn ®, see 3. toxicity test) | 10.00 |
| Alcohol | 7.80 |
| Elestab ® 50 J | 0.27 |
| PEG-40 Hydrogenated Castor Oil | 0.30 |
| Perfumes | 0.10 |
| Acrylates/Steareth-20 Itaconate Copolymer | 1.25 |
| NaOH (N) | 1.50 |

In the placebo lotion, the active ingredient was replaced by water.

Results:

TABLE 5a
% of telogenic hairs on the frontal side after treatment for
2, 4 and 6 months based on the value at the beginning of the
study on day 0 (=100), mean value: n = 15 volunteers

| | Day 0 | Day 56 | Day 112 | Day 168 |
|---|---|---|---|---|
| Placebo lotion | 100 | 120 | 118 | 110 |
| Lotion containing | 100 | 99 | 85 | 101 |
| 10% by weight | | | | |
| Trichodyn ® | | | | |

TABLE 5b
% of telogenic hairs on the occipital side after treatment for
2, 4 and 6 months based on the value at the beginning of the study
on day 0 (=100), mean value: n = 15 volunteers

| | Day 0 | Day 56 | Day 112 | Day 168 |
|---|---|---|---|---|
| Placebo lotion | 100 | 120 | 118 | 110 |
| Lotion containing | 100 | 99 | 85 | 101 |
| 10% by weight | | | | |
| Trichodyn ® | | | | |

After treatment with placebo lotion for 6 months, the percentage of hairs in the telogenic phase had significantly increased in both regions of the test scalps, but especially in the occipital region, which corresponds to the normal increase in alopecia in summer. After treatment with Trichodyn®, however, there was a distinct reduction in hair loss, i.e. the hair remained in place.

8. Exemplary Formulations of Cosmetic Preparations containing Trichodyn®

The preparations according to the invention were used in the following formulations K1 to K21 and 1 to 30 according to the invention.

The cosmetic preparations thus produced showed very good skin care properties in relation to comparison formulations C1, C2 and C3 coupled with good dermatological compatibility. In addition, the preparations according to the invention are stable to oxidative decomposition.

TABLE 6
Soft cream formulations K1 to K7 (All quantities in
% by weight, based on the cosmetic preparation)

| INCI name | K1 | K2 | K3 | K4 | K5 | K6 | K7 | C1 |
|---|---|---|---|---|---|---|---|---|
| Glyceryl Stearate | 8.0 | 8.0 | 8.0 | 8.0 | 8.0 | 8.0 | 8.0 | 8.0 |
| (and) Ceteareth- | | | | | | | | |
| 12/20 (and) | | | | | | | | |
| Cetearyl Alcohol | | | | | | | | |
| (and) Cetyl | | | | | | | | |
| Palmitate | | | | | | | | |
| Cetearyl Alcohol | 2.0 | 2.0 | 2.0 | 2.0 | 2.0 | 2.0 | 2.0 | 2.0 |
| Dicaprylyl Ether | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl | 3.0 | 3.0 | 3.0 | 3.0 | 3.0 | 3.0 | 3.0 | 3.0 |
| Isononanoate | | | | | | | | |
| Glycerin | 3.0 | 3.0 | 3.0 | 3.0 | 3.0 | 3.0 | 3.0 | 3.0 |
| (86% by weight) | | | | | | | | |
| Trichodyn ® | 0.5 | 0.5 | 0.5 | 0.5 | 0.5 | 0.5 | 0.5 | — |
| Tocopherol | | 0.5 | | | | | | |
| Allantoin | | | 0.2 | | | | | |
| Bisabolol | | | | 0.5 | | | | |
| Chitosan | | | | | 10.0 | | | |
| (Hydagen CMF) | | | | | | | | |
| Deoxyribonucleic | | | | | | 0.5 | | |
| acid¹⁾ | | | | | | | | |
| Panthenol | | | | | | | 0.5 | |

| | |
|---|---|
| Water | to 100 |

TABLE 7
Night cream formulations K8 to K14 (All quantities
in % by weight, based on the cosmetic preparation)

| INCI name | K8 | K9 | K10 | K11 | K12 | K13 | K14 | C2 |
|---|---|---|---|---|---|---|---|---|
| Polyglyceryl-2 | 4.0 | 4.0 | 4.0 | 4.0 | 4.0 | 4.0 | 4.0 | 5.0 |
| Dipolyhydroxy- | | | | | | | | |
| stearate | | | | | | | | |
| Polyglyceryl-3 | 2.0 | 2.0 | 2.0 | 2.0 | 2.0 | 2.0 | 2.0 | 2.0 |
| Diisostearate | | | | | | | | |
| Cera Alba | 2.0 | 2.0 | 2.0 | 2.0 | 2.0 | 2.0 | 2.0 | 2.0 |
| Zinc Stearate | 2.0 | 2.0 | 2.0 | 2.0 | 2.0 | 2.0 | 2.0 | 2.0 |
| Cocoglycerides | 3.0 | 3.0 | 3.0 | 3.0 | 3.0 | 3.0 | 3.0 | 3.0 |
| Cetearyl | 8.0 | 8.0 | 8.0 | 8.0 | 8.0 | 8.0 | 8.0 | 8.0 |
| Isononanoate | | | | | | | | |
| Dicaprylyl Ether | 5.0 | 5.0 | 5.0 | 5.0 | 5.0 | 5.0 | 5.0 | 5.0 |
| Magnesium sulfate | 1.0 | 1.0 | 1.0 | 1.0 | 1.0 | 1.0 | 1.0 | 1.0 |
| Glycerin (86% | 5.0 | 5.0 | 5.0 | 5.0 | 5.0 | 5.0 | 5.0 | 5.0 |
| by weight) | | | | | | | | |
| Trichodyn ® | 0.5 | 0.5 | 0.5 | 0.5 | 0.5 | 0.5 | 0.5 | — |
| Tocopherol | | 0.5 | | | | | | |
| Allantoin | | | 0.2 | | | | | |
| Bisabolol | | | | 0.5 | | | | |
| Chitosan | | | | | 10.0 | | | |
| (Hydagen CMF) | | | | | | | | |
| Deoxyribo- | | | | | | 0.5 | | |
| nucleic acid¹⁾ | | | | | | | | |
| Panthenol | | | | | | | 0.5 | |

| | |
|---|---|
| Water | to 100 |

TABLE 8
W/O body lotion formulations K15 to K21. (All quantities
in % by weight, based on the cosmetic preparation)

| INCI name | K15 | K16 | K17 | K18 | K19 | K20 | K21 | C3 |
|---|---|---|---|---|---|---|---|---|
| PEG-7 Hydro- | 7.0 | 7.0 | 7.0 | 7.0 | 7.0 | 7.0 | 7.0 | 7.0 |
| genated | | | | | | | | |
| Castor Oil | | | | | | | | |
| Decyl Oleate | 7.0 | 7.0 | 7.0 | 7.0 | 7.0 | 7.0 | 7.0 | 7.0 |
| Cetearyl | 7.0 | 7.0 | 7.0 | 7-0 | 7.0 | 7.0 | 7.0 | 7.0 |
| Isononanoate | | | | | | | | |
| Glycerin (86% | 5.0 | 5.0 | 5.0 | 5.0 | 5.0 | 5.0 | 5.0 | 5.0 |
| by weight) | | | | | | | | |
| MgSO₄ · 7H₂O | 1.0 | 1.0 | 1.0 | 1.0 | 1.0 | 1.0 | 1.0 | 1.0 |
| Trichodyn ® | 1.5 | 1.5 | 1.5 | 1.5 | 1.5 | 1.5 | 1.5 | — |
| Tocopherol | | 0.5 | | | | | | |
| Allantoin | | | 0.2 | | | | | |
| Bisabolol | | | | 0.5 | | | | |
| Chitosan | | | | | 10.0 | | | |
| (Hydagenc | | | | | | | | |
| CMF) | | | | | | | | |
| Deoxyribo- | | | | | | 0.5 | | |
| nucleic | | | | | | | | |
| acid¹⁾ | | | | | | | | |
| Panthenol | | | | | | | 0.5 | |

| | |
|---|---|
| Water | to 100 |

¹⁾Deoxyribonucleic acid: molecular weight ca. 70,000, purity (determined by spectrophotometric measurement of absorption at 260 nm and 280 nm): at least 1.7

TABLE 9
Cosmetic preparations (all quantities in % by
weight, based on the cosmetic preparation, water,
preservative add up to 100% by weight)

| | 1 | 2 | 3 | 4 |
|---|---|---|---|---|
| | % by | % by | % by | % by |
| Composition (INCI) | wt. | wt. | wt. | wt. |
| Texapon ® NSO | 38.0 | 38.0 | 25.0 | — |
| Sodium Laureth Sulfate | | | | |
| Texapon ® SB 3 | — | — | 10.0 | — |
| Disodium Laureth | | | | |
| Sulfosuccinate | | | | |
| Plantacare ® 818 | 7.0 | 7.0 | 6.0 | — |
| Coco Glucosides | | | | |
| Plantacare ® PS 10 | — | — | — | 20.0 |
| Sodium Laureth Sulfate | | | | |
| (and) Coco Glucosides | | | | |
| Dehyton ® PK 45 | — | — | 10.0 | — |
| Cocamidopropyl Betaine | | | | |
| Lamesoft ® PO 65 | 3.0 | | | 4.0 |
| Coco-Glucoside (and) | | | | |
| Glyceryl Oleate | | | | |
| Lamesoft ® LMG | — | 5.0 | — | — |
| Glyceryl Laurate (and) | | | | |
| Potassium Cocoyl | | | | |
| Hydrolyzed Collagen | | | | |
| Euperlan ® PK 3000 AM | — | 3.0 | 5.0 | 5.0 |
| Glycol Distearate (and) | | | | |
| Laureth-4 (and) | | | | |
| Cocamidopropyl Betaine | | | | |
| Trichodyn ® | 1.0 | 1.0 | 1.0 | 1.0 |
| Arlypon ® F | 3.0 | 3.0 | 1.0 | — |
| Laureth-2 | | | | |
| Sodium Chloride | — | 1.5 | — | 1.5 |

(1-2) shower bath,
(3) shower gel,
(4) wash lotion

TABLE 10
Cosmetic preparations “2-in-1” shower bath (all
quantities in % by weight, based on the cosmetic preparation,
water, preservative add up to 100% by weight)

| Composition (INCI) | 5 | 6 | 7 | 8 |
|---|---|---|---|---|
| Texapon ® NSO | 30.0 | 25.0 | | 25.0 |
| Sodium Laureth Sulfate | | | | |
| Plantacare ® 818 | | | | 8.0 |
| Coco Glucosides | | | | |
| Plantacare ® 2000 | | 8.0 | | |
| Decyl Glucoside | | | | |
| Plantacare ® PS 10 | | | 20.0 | |
| Sodium Laureth Sulfate | | | | |
| (and) Coco Glucosides | | | | |
| Dehyton ® PK 45 | | 10.0 | 10.0 | |
| Cocamidopropyl Betaine | | | | |
| Lamesoft ® PO 65 | 5.0 | | | |
| Coco-Glucoside (and) | | | | |
| Glyceryl Oleate | | | | |
| Lamesoft ® LMG | | 5.0 | 5.0 | |
| Glyceryl Laurate (and) | | | | |
| Potassium Cocoyl | | | | |
| Hydrolyzed Collagen | | | | |
| Gluadin ® WQ | 3.0 | | | |
| Laurdimonium Hydroxypropyl | | | | |
| Hydrolyzed Wheat Protein | | | | |
| Gluadin ® WK | | | | |
| Sodium Cocoyl Hydrolyzed | | | | |
| Wheat Protein | | | | |
| Euperlan ® PK 3000 AM | 5.0 | 3.0 | 4.0 | — |
| Glycol Distearate (and) | | | | |
| Laureth-4 (and) | | | | |
| Cocamidopropyl Betaine | | | | |
| Panthenol | 0.5 | — | — | 0.5 |
| Trichodyn ® | 1.0 | 1.0 | 1.0 | 1.0 |
| Arlypon ® F | 2.6 | 1.6 | — | 1.0 |
| Laureth-2 | | | | |
| Sodium Chloride | — | — | — | — |

TABLE 11
Cosmetic preparations (all quantities in % by weight, based on the
cosmetic preparation, water, preservative add up to 100% by weight)

| Composition (INCI) | 9 | 10 | 11 | 12 | 13 | 14 | 15 | 16 | 17 | 18 |
|---|---|---|---|---|---|---|---|---|---|---|
| Dehymuls ® PGPH | 4.0 | 3.0 | — | 5.0 | — | — | — | — | — | — |
| Polyglyceryl-2 Dipolyhydroxystearate | | | | | | | | | | |
| Lameform ® TGI | 2.0 | 1.0 | — | — | — | — | — | — | — | — |
| Polyglyceryl-3 Diisostearate | | | | | | | | | | |
| Emulgade ® PL 68/50 | — | — | — | — | 4.0 | — | — | — | 3.0 | — |
| Cetearyl Glucoside (and) Cetearyl Alcohol | | | | | | | | | | |
| Eumulgin ® B2 | — | — | — | — | — | — | — | 2.0 | — | — |
| Ceteareth-20 | | | | | | | | | | |
| Tegocare ® PS | — | — | 3.0 | — | — | — | 4.0 | — | — | — |
| Polyglyceryl-3 Methylglucose Distearate | | | | | | | | | | |
| Eumulgin VL 75 | — | — | — | — | — | 3.5 | — | — | 2.5 | — |
| Polyglyceryl-2 Dipolyhydroxystearate (and) | | | | | | | | | | |
| Lauryl Glucoside (and) Glycerin | | | | | | | | | | |
| Bees Wax | 3.0 | 2.0 | 5.0 | 2.0 | — | — | — | — | — | — |
| Cutina ® GMS | — | — | — | — | — | 2.0 | 4.0 | — | — | 4.0 |
| Glyceryl Stearate | | | | | | | | | | |
| Lanette ® O | — | — | 2.0 | — | 2.0 | 4.0 | 2.0 | 4.0 | 4.0 | 1.0 |
| Cetearyl Alcohol | | | | | | | | | | |
| Antaron ® V 216 | — | — | — | — | — | 3.0 | — | — | — | 2.0 |
| PVP/Hexadecene Copolymer | | | | | | | | | | |
| Myritol ® 818 | 5.0 | — | 10.0 | — | 8.0 | 6.0 | 6.0 | — | 5.0 | 5.0 |
| Cocoglycerides | | | | | | | | | | |
| Finsolv ® TN | — | 6.0 | — | 2.0 | — | — | 3.0 | — | — | 2.0 |
| C12/15 Alkyl Benzoate | | | | | | | | | | |
| Cetiol ® J 600 | 7.0 | 4.0 | 3.0 | 5.0 | 4.0 | 3.0 | 3.0 | — | 5.0 | 4.0 |
| Oleyl Erucate | | | | | | | | | | |
| Cetiol ® OE | 3.0 | — | 6.0 | 8.0 | 6.0 | 5.0 | 4.0 | 3.0 | 4.0 | 6.0 |
| Dicaprylyl Ether | | | | | | | | | | |
| Mineral Oil | — | 4.0 | — | 4.0 | — | 2.0 | — | 1.0 | — | — |
| Cetiol ® PGL | — | 7.0 | 3.0 | 7.0 | 4.0 | — | — | — | 1.0 | — |
| Hexadecanol (and) Hexyldecyl Laurate | | | | | | | | | | |
| Panthenol/Bisabolol | 1.2 | 1.2 | 1.2 | 1.2 | 1.2 | 1.2 | 1.2 | 1.2 | 1.2 | 1.2 |
| Trichodyn ® | 1.0 | 1.0 | 1.0 | 1.0 | 1.0 | 1.0 | 1.0 | 1.0 | 1.0 | 1.0 |
| Copherol ® F 1300 | 0.5 | 1.0 | 1.0 | 2.0 | 1.0 | 1.0 | 1.0 | 2.0 | 0.5 | 2.0 |
| Tocopherol/Tocopheryl Acetate | | | | | | | | | | |
| Neo Heliopan ® Hydro | 3.0 | — | — | 3.0 | — | — | 2.0 | — | 2.0 | — |
| Sodium Phenylbenzimidazole Sulfonate | | | | | | | | | | |
| Neo Heliopan ® 303 | — | 5.0 | — | — | — | 4.0 | 5.0 | — | — | 10.0 |
| Octocrylene | | | | | | | | | | |
| Neo Heliopan ® BB | 1.5 | — | — | 2.0 | 1.5 | — | — | — | 2.0 | — |
| Benzophenone-3 | | | | | | | | | | |
| Neo Heliopan ® E 1000 | 5.0 | — | 4.0 | — | 2.0 | 2.0 | 4.0 | 10.0 | — | — |
| Isoamyl p-Methoxycinnamate | | | | | | | | | | |
| Neo Heliopan ® AV | 4.0 | — | 4.0 | 3.0 | 2.0 | 3.0 | 4.0 | — | 10.0 | 2.0 |
| Octyl Methoxycinnamate | | | | | | | | | | |
| Uvinul ® T 150 | 2.0 | 4.0 | 3.0 | 1.0 | 1.0 | 1.0 | 4.0 | 3.0 | 3.0 | 3.0 |
| Octyl Triazone | | | | | | | | | | |
| Zinc Oxide | — | 6.0 | 6.0 | — | 4.0 | — | — | — | — | 5.0 |
| Titanium Dioxide | — | — | — | — | — | — | — | 5.0 | — | — |
| Glycerol (86% by weight) | 5.0 | 5.0 | 5.0 | 5.0 | 5.0 | 5.0 | 5.0 | 5.0 | 5.0 | 5.0 |

(14) w/o sun protection cream,
(15-17) w/o sun protection lotion,
(18, 21, 23) o/w sun protection lotion,
(19, 20, 22) o/w sun protection cream

TABLE 12
Cosmetic preparations (all quantities in % by
weight, based on the cosmetic preparation, water,
preservative add up to 100% by weight)

| | 19 | 20 | 21 | 22 | 23 | 24 |
|---|---|---|---|---|---|---|
| | % by | % by | % by | % by | % by | % by |
| Composition (INCI) | wt. | wt. | wt. | wt. | wt. | wt. |
| Dehyquart ® A | 4.0 | 4.0 | | | 3.0 | |
| Cetrimonium Chloride | | | | | | |
| Dehyquart L ® 80 | | | 1.2 | 1.2 | | 1.0 |
| Dococoylmethylethoxy- | | | | | | |
| monium Methosulfate | | | | | | |
| (and) Propyleneglycol | | | | | | |
| Eumulgin ® B2 | 0.8 | | — | 0.8 | — | 1.0 |
| Ceteareth-20 | | | | | | |
| Eumulgin ® VL 75 | — | 2.0 | 2.0 | — | 0.8 | — |
| Lauryl Glucoside (and) | | | | | | |
| Polyglyceryl-2 | | | | | | |
| Polyhydroxystearate | | | | | | |
| (and) Glycerin | | | | | | |
| Lanette ® O | 3.0 | 3.0 | 3.0 | 3.0 | 3.0 | 3.0 |
| Cetearyl Alcohol | | | | | | |
| Cutina ® GMS | — | 0.5 | — | 0.5 | — | 1.0 |
| Glyceryl Stearate | | | | | | |
| Lamesoft ® PO 65 | | — | 3.0 | — | — | 3.0 |
| Coco-Glucoside (and) | | | | | | |
| Gyceryl Oleate | | | | | | |
| Cetiol ® J 600 | — | 0.5 | — | 1.0 | — | 1.0 |
| Oleyl Erucate | | | | | | |
| Eutanol ® G | — | — | 1.0 | — | — | 1.0 |
| Octyldodecanol | | | | | | |
| Nutrilan ® Keratin W | 5.0 | — | — | 2.0 | — | — |
| Hydrolyzed Keratin | | | | | | |
| Generol ® 122 N | — | — | — | — | 1.0 | 1.0 |
| Soya Sterol | | | | | | |
| Trichodyn ® | 1.0 | 1.0 | 1.0 | 1.0 | 1.0 | 1.0 |
| Copherol ® 1250 | — | — | 0.1 | 0.1 | — | — |
| Tocopheryl Acetate | | | | | | |

(24-27) Hair rinse,
(28-29) hair treatment

TABLE 13
Cosmetic preparations shampoo (all quantities in
% by weight, based on the cosmetic preparation,
water, preservative add up to 100% by weight)

| Composition (INCI) | 25 | 26 | 27 | 28 | 29 | 30 |
|---|---|---|---|---|---|---|
| Texapon ® NSO | 30.0 | | | 30.0 | 25.0 | |
| Sodium Laureth Sulfate | | | | | | |
| Texapon ® K 14 S | | 30.0 | | | | 30.0 |
| Sodium Myreth Sulfate | | | | | | |
| Texapon ® SB 3 | | 10.0 | | | | |
| Disodium Laureth | | | | | | |
| Sulfosuccinate | | | | | | |
| Plantacare ® 818 | 4.0 | | | | | |
| Coco Glucosides | | | | | | |
| Plantacare ® 2000 | | 4.0 | | | | |
| Decyl Glucoside | | | | | | |
| Plantacare ® PS 10 | | | 20.0 | | | |
| Sodium Laureth Sulfate | | | | | | |
| (and) Coco Glucosides | | | | | | |
| Dehyton ® PK 45 | 5.0 | | | 10.0 | | 10.0 |
| Cocamidopropyl Betaine | | | | | | |
| Gluadin ® WK | | | | | 8.0 | |
| Sodium Cocyl Hydrolyzed | | | | | | |
| Wheat Protein | | | | | | |
| Lamesoft ® PO 65 | — | — | — | — | 2.0 | 2.0 |
| Coco-Glucoside (and) | | | | | | |
| Glyceryl Oleate | | | | | | |
| Nutrilan ® Keratin W | 5.0 | — | — | — | — | — |
| Hydrolyzed Keratin | | | | | | |
| Gluadin ® W 40 | — | 2.0 | — | 2.0 | — | — |
| Hydrolyzed Wheat Protein | | | | | | |
| Euperlan ®PK 3000 AM | — | — | — | 3.0 | 3.0 | — |
| Glycol Distearate (and) | | | | | | |
| Laureth-4 (and) | | | | | | |
| Cocamidopropyl Betaine | | | | | | |
| Panthenol | — | — | — | — | — | 0.2 |
| Trichodyn ® | 1.0 | 1.0 | 1.0 | 1.0 | 1.0 | 1.0 |
| Arlypon ® F | 1.5 | — | — | — | — | — |
| Laureth-2 | | | | | | |
| Sodium Chloride | — | 1.6 | 2.0 | 2.2 | — | 3.0 |

All substances with the registered trade mark symbol ® used and listed in Tables 6 to 13 are marks and products of the COGNIS Group.

1. Cosmetic and/or pharmaceutical preparations containing
(a) an effective quantity of an extract of Pterocarpus marsupium and
(b) dicarboxylic acids and/or salts thereof and/or amino acids.

2. Preparations as claimed in claim 1, characterized in that the extracts exclusively or predominantly contain flavone derivatives as active ingredients.

3. Preparations as claimed in claim 1, characterized in that they contain dicarboxylic acids selected from the group consisting of oxalic acid, malonic acid, succinic acid and glutamic acid.

4. Preparations as claimed in claim 1 or 2, characterized in that they contain salts of the dicarboxylic acids mentioned in claim 3 selected from the group consisting of alkali metal and alkaline earth metal salts, more particularly sodium, potassium magnesium and calcium salts.

5. Preparations as claimed in at least one of claims 1 to 4, characterized in that they contain amino acids selected from the group consisting of glycine, alanine, leucine, isoleucine, serine, threonine, cysteine, aspartic acid, glutamic acid, asparagine, glutamine, phenylalanine, tyrosine, methionine, valine, proline, lysine and histidine.

6. Preparations as claimed in at least one of claims 1 to 6, characterized in that they contain
(a) 0.001 to 25% by weight extract and
(b) 0.001 to 15% by weight dicarboxylic acids and/or salts thereof and/or 0.0005 to 10% by weight amino acids,
with the proviso that the quantities shown optionally add up to 100% by weight with water and/or other auxiliaries and additives.

7. The use of the preparations claimed in at least one of claims 1 to 6 as hair and skin care preparations.

8. The use of the preparations claimed in at least one of claims 1 to 6 against hair loss, more particularly against androgenic alopecia.

9. The use of the preparations claimed in at least one of claims 1 to 6 as preparations with anti-glycosylation activity.

10. The use of the preparations claimed in at least one of claims 1 to 6 for stimulating the synthesis of glutathione.

11. The use of the preparations claimed in at least one of claims 1 to 6 as anti-inflammatory components.

12. The use of the preparations claimed in at least one of claims 1 to 6 for reducing the formation of interleucine-1-alpha (1′IL-α).

13. The use of the preparations claimed in at least one of claims 1 to 6 against the ageing of skin.

14. The use of the preparations claimed in at least one of claims 1 to 6 as UV/IR protection factors.

15. The use of the preparations claimed in at least one of claims 1 to 6 against the damaging of fibroblasts and/or keratinocytes by UV radiation.

16. The use of the preparations claimed in at least one of claims 1 to 6 against oxidative skin and/or hair stress.

17. The use of the preparations claimed in at least one of claims 1 to 6 in protective and restorative care preparations with revitalizing and reactivating activity for the skin and especially the scalp.

18. The use of Pterocarpus marsupium extracts against hair loss, more particularly against androgenic alopecia.

19. The use of Pterocarpus marsupium extracts as preparations with anti-glycosylation activity.

20. The use of Pterocarpus marsupium extracts for stimulating the synthesis of glutathione.

21. The use of Pterocarpus marsupium extracts for reducing the formation of interleucine-1 -alpha (1′IL-α).

22. The use of Pterocarpus marsupium extracts against the ageing of skin.

23. The use of Pterocarpus marsupium extracts as UV/IR protection factors.

24. The use of Pterocarpus marsupium extracts as active components against the damaging of fibroblasts and/or keratinocytes by UV radiation.

25. The use of Pterocarpus marsupium extracts against oxidative skin and/or hair stress.

26. The use of Pterocarpus marsupium extracts in protective and restorative care preparations with revitalizing and reactivating activity for the skin and especially the scalp.
